(19) United States
(12) Patent Application Publication
(10) Pub. No.: US 2009/0062732 A1
(43) Pub. Date: Mar. 5, 2009

(54) Connectable Device and a Device Assembly for Delivering a Liquid Drug

(75) Inventor: Bo Radmer, Hillerod (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/282,927

(22) PCT Filed: Feb. 6, 2007
(86) PCT No.: PCT/EP2007/051115

§ 371 Date: Oct. 28, 2008

(30) Foreign Application Priority Data
Mar. 17, 2006 (EP) 06005478.0
Aug. 18, 2006 (EP) 06119150.8

(51) Int. Cl. A61M 5/31

(52) U.S. Cl. 604/84

(57) ABSTRACT

A device (1) for delivering a liquid drug to a user. The device (1) comprises a reservoir (4, 20) adapted to contain a liquid, an outlet opening (7) for delivering liquid drug from the device (1), and means for connecting the device (1) to at least one other at least substantially identical device (1). The devices (1) are connected in such a manner that each of the devices (1) is in fluid communication with a common outlet (19), the common outlet (19) thereby being adapted to deliver liquid drug from each of the devices (1). In one embodiment the devices (1) may be connected to an external operating unit (14), and operational forces from the operating unit (14) may be transferred between the devices (1). Thereby the external operating unit (14) may operate all of the connected devices (1) simultaneously. The device (1) is further adapted to perform reconstitution of a dry drug, such as a lyophilized drug, contained in a vial (2). The device (1) may be used as a ‘building block’ for constructing a drug delivery device using an appropriate number of devices (1) in order to deliver a desired dose of drug. Furthermore, a device assembly (10) comprising at least two connected devices (1).

### FIELD OF THE INVENTION

The present invention relates to a device for delivering a liquid drug to a user, the device being connectable to at least one other at least substantially identical device. The present invention further relates to an assembly of at least two such devices being interconnected.

### BACKGROUND OF THE INVENTION

When two or more vials with fluid intravenous (IV) drug are needed for one infusion, it is necessary to change the connection between two syringes during the infusion while the needle delivering the drug is penetrating the skin of the person receiving the drug. The user will have to handle the needle tube very carefully while the skin is penetrated in order to avoid that the needle is pulled out of the vein during infusion. Since the drug will normally not have the desired effect if it is delivered outside a vein, it is imperative that the needle remains in the vein during the complete infusion.

In order to overcome the problems described above, systems for pooling the contents of two or more vials have been provided. In such systems the drug from each of the vials constituting the desired dose is collected in a common reservoir, container or syringe, the common reservoir, container or syringe having a sufficiently large volume to hold the entire dose of drug. The dose is subsequently delivered from the common reservoir, container or syringe. It is a drawback of such pooling systems that it is necessary to perform a number of steps prior to delivering the desired dose. Thus, it is necessary to collect the drug from the desired number of separate vials and apply it sequentially to the common reservoir, container or syringe. This is cumbersome, and in some pooling systems there is also a risk of needle sticks and/or contamination of the drug and/or various surfaces of the vials/containers involved. It is therefore desirable to be able to solve the problems described above without having to perform additional steps.

### SUMMARY OF THE INVENTION

It is, thus, an object of the invention to provide a device for delivering a drug which in a simple manner allows collecting of a dose of drug which exceeds the contents of a single vial.

It is a further object of the invention to provide a device for delivering a drug which reduces the risk of contamination when collecting a dose of drug which exceeds the contents of a single vial.

It is an even further object of the invention to provide a device for delivering a drug providing the possibility of constructing a device being capable of delivering a desired dose of liquid drug.

It is an even further object of the invention to provide a device assembly for delivering a dose of drug which exceeds the contents of a single vial.

According to a first aspect of the invention the above and other objects are fulfilled by providing a device for delivering a liquid drug to a user, the device comprising:
a reservoir adapted to contain a liquid,
a container containing a dry drug,
means for establishing a fluid connection between the container and the reservoir in order to allow liquid to pass from the reservoir to the container in order to provide liquid drug,
an outlet opening for delivering liquid drug from the device, and
means for connecting the device to at least one other at least substantially identical device in such a manner that each of the devices is in fluid communication with a common outlet, said common outlet thereby being adapted to deliver liquid drug from each of the devices.

The reservoir may be any suitable kind of reservoir adapted to contain a liquid, including a vial, a container, a flexible reservoir, such as a bag, a syringe, etc.

The device further comprises a container containing a dry drug and means for establishing a fluid connection between the container and the reservoir in order to allow liquid to pass from the reservoir to the container in order to provide liquid drug. Thus, the drug is initially in a dry form, i.e. it must be reconstituted before it can be delivered. A dry drug may, e.g., be a lyophilized drug, a powder, a tablet, a granulate, etc. When the drug is reconstituted, a liquid is applied to the container containing the dry drug, and the drug is solved in the liquid, thereby forming a liquid drug. The liquid drug may then be delivered to the person to receive the drug. The liquid drug may be delivered directly from the container. In this case the device is preferably adapted to be connected to another at least substantially identical device in such a manner that each of the containers is in fluid communication with the common outlet.

As an alternative, the liquid drug may be retrieved to the reservoir after reconstitution and prior to delivery of the liquid drug. In this case the device is preferably adapted to be connected to another at least substantially identical device in such a manner that each of the reservoirs is in fluid communication with the common outlet.

Thus, the liquid is initially contained in the reservoir. When it is desired to deliver a dose of drug, the fluid connection between the container and the reservoir is established, and the liquid is transferred to the container. When the drug has been reconstituted, the reconstituted drug may be retrieved to the reservoir. The reconstitution of the drug may be performed before the desired number of devices are connected, or it may be performed while the devices are connected.

The outlet opening forms a passage between the interior and the exterior of the device. The outlet opening must be in fluid connection with a part of the device containing the liquid drug, e.g. the container or the reservoir as described above. Alternatively, it must at least be possible to establish such a connection, in order to allow liquid drug contained in the device to be delivered from the device via the outlet opening.

The device comprises means for connecting the device to at least one other at least substantially identical device. The other device(s) should be identical to the device in the sense that it/they must be connectable to a device according to the first aspect of the invention, and in the sense that it/they must comprise a part, e.g. a container or a reservoir, adapted to contain a liquid drug. Preferably, the devices are identical, but there may be differences as long as these differences do not affect the connectability of the devices. When the device is connected to at least one other device, each of the devices is in fluid communication with a common outlet. Thereby this common outlet may deliver liquid drug from each of the devices, either sequentially or as one combined dose of drug.

It is an advantage that the device is connectable to at least one other at least substantially identical device, because this makes the device very flexible in the sense that a dose of liquid drug having a desired size may be formed, merely be selecting the number of devices which are connected. Thus, in case a relatively small dose of drug is desired, a single device may be used, i.e. the device may be used on its own in an ordinary manner, without making use of the connecting means. However, the device may also be used in combination with one, two, three, four, or any other desirable number of other devices to form a double, triple, etc. dose of liquid drug, deliverable from one combined device formed by the connected individual devices. Thus, the device may be used as a ‘building block’ for customizing a delivery device for forming a desired dose of drug. Accordingly, a dose of liquid drug having a desired size can be delivered without having to change device or reservoir while an infusion needle is penetrating the skin of a person receiving the dose.

The device may further comprise an inlet opening being connectable to an outlet opening of another at least substantially identical device. In this case an outlet opening may be connected to the inlet opening of an adjacent device, thereby connecting the devices in series. Accordingly, there is no limit to the number of devices which may be interconnected in this manner.

The device may further comprise means for connecting the device to an external operating unit, and means for transferring operational forces from the external operating unit to another at least substantially identical device connected to the device, the external operating unit thereby being capable of operating the device as well as the device connected thereto. Thereby a number of interconnected devices may all be operated by means of a single external operating unit. This may, e.g., be desirable in case the devices are pump driven, thereby making them easier to operate for persons with poor motoric skills and/or low dexterity, e.g. elderly persons or children. According to this embodiment it is only necessary to provide a single external operating unit to operate all of the devices. Such an operating unit will typically be reusable, while the devices will be disposable.

The means for transferring operational forces may comprise a mechanical, pneumatic or hydraulic force transferring mechanism. A mechanical transferring mechanism may comprise a physical connection, such as a drive shaft, or a linear actuator connected via a wedge. A pneumatic or hydraulic force transferring mechanism may comprise means for pressing a piston in a cylinder, thereby forcing liquid, e.g. from the reservoir to a vial containing a dry drug, or forcing liquid drug from the reservoir towards the common outlet. The piston may, e.g., be pressed by means of pressurized air or liquid.

Alternatively or additionally, the means for transferring operational forces may be adapted to transfer a torque. This may, e.g., be in the form of a rotational shaft which is adapted to engage an at least substantially identical rotational shaft positioned on another device, in such a manner that a rotational movement of one of the shafts results in a corresponding rotational movement of the engaging shaft. This may, e.g., be obtained by means of a set of mating drive surfaces or a set of mating teeth positioned on each of the shafts.

Thus, the means for transferring operational forces may comprise at least one drive shaft being adapted to engage with a drive shaft positioned on another at least substantially identical device.

Alternatively, the means for transferring operational forces may be adapted to transfer a linear movement.

The external operating unit may comprise a motor, e.g. an electromotor, a hydraulic motor, a pneumatic motor, or a mechanical motor, such as a spring driven motor. A motor may advantageously be used for driving drive shafts.

Alternatively, the operating unit may comprise pressure generating means, e.g. a gas cartridge, which may operate the devices. Or the operating unit may comprise a spring, in which a tension can be build up. When the tension is subsequently released, rotational or linear forces may thereby be transferred to the devices. Or the operating unit may comprise a battery, and the battery may be used for driving separate motors positioned in each of the devices.

According to a second aspect of the invention the above and other objects are fulfilled by providing a device assembly comprising at least two devices according to the first aspect of the invention, said devices being interconnected in such a manner that liquid drug can be delivered from each of the devices via a common outlet opening.

It should be noted that a person skilled in the art would readily recognise that any feature described in combination with the first aspect of the invention may also be combined with the second aspect of the invention, and vice versa.

As described above, the devices may be connected in series.

The device assembly may further comprise means for connecting to an external operating unit, and the connection between the devices may comprise means for transferring operational forces between the devices, an operational force from the external operating unit thereby being transferable to each of the devices. This has also been described above.

The device assembly may further comprise drug delivery means for delivering a drug from the common outlet to a user. The drug delivery means may comprise a common reservoir being detachably mounted at the common outlet. In this case the combined dose of drug originating from the connected devices is collected in the common reservoir, via the common outlet. The common reservoir is then detached from the device assembly, and the dose is subsequently delivered from the common reservoir. The common reservoir may be a syringe, a flexible reservoir, such as a bag, or any other suitable kind of reservoir. When the common reservoir has been detached from the device assembly, a needle may advantageously be attached at an inlet/outlet opening which was connecting the common reservoir to the device assembly while the drug was collected in the common reservoir. The common reservoir and the needle may be connected via one or more tubes.

Alternatively, the drug delivery means may comprise an infusion needle, e.g. a butterfly needle, attached directly to the device assembly, possibly via one or more tubes. In this case the drug is delivered directly from the device assembly to the person receiving the drug.

### BRIEF DESCRIPTION OF THE DRAWINGS

The invention will now be described in further details with reference to the accompanying drawings in which

FIG. 1 is a perspective view of a device according to an embodiment of the invention,

FIG. 2 is a perspective view of three devices of FIG. 1 connected to form a device assembly according to an embodiment of the invention,

FIG. 3 is a perspective view of a device assembly according to an embodiment of the invention and a box for storing the device assembly,

FIG. 4 is a perspective view of a box for storing a device,

FIG. 5 is a perspective view of a device assembly according to an embodiment of the invention and having an operating unit coupled thereto,

FIG. 6 is a perspective view of a device assembly according to an alternative embodiment of the invention, and

FIG. 7 is a perspective view of a device assembly according to yet another embodiment of the invention.

### DETAILED DESCRIPTION OF THE DRAWINGS

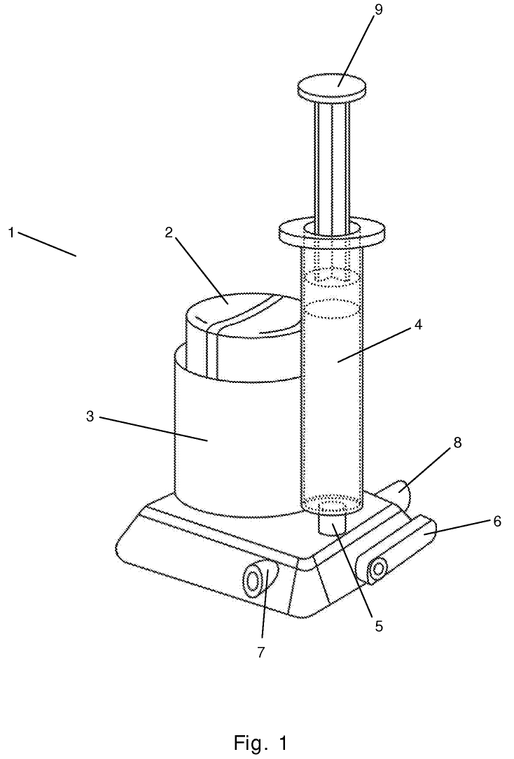

FIG. 1 is a perspective view of a device 1 for delivering a liquid drug to a person. The device 1 comprises a vial 2 positioned in a vial adapter 3 and a syringe 4 positioned in a syringe adapter 5. The vial 2 contains a dry drug, e.g. a lyophilized drug, and the syringe 4 contains a liquid which is suitable for reconstituting the dry drug contained in the vial 2. The device 1 is further provided with a selector 6 which is operable to establish or interrupt fluid connections between the vial 2 and the syringe 4, between the syringe 4 and an outlet opening 7, and between an inlet opening 8 and the outlet opening. This will be described further below.

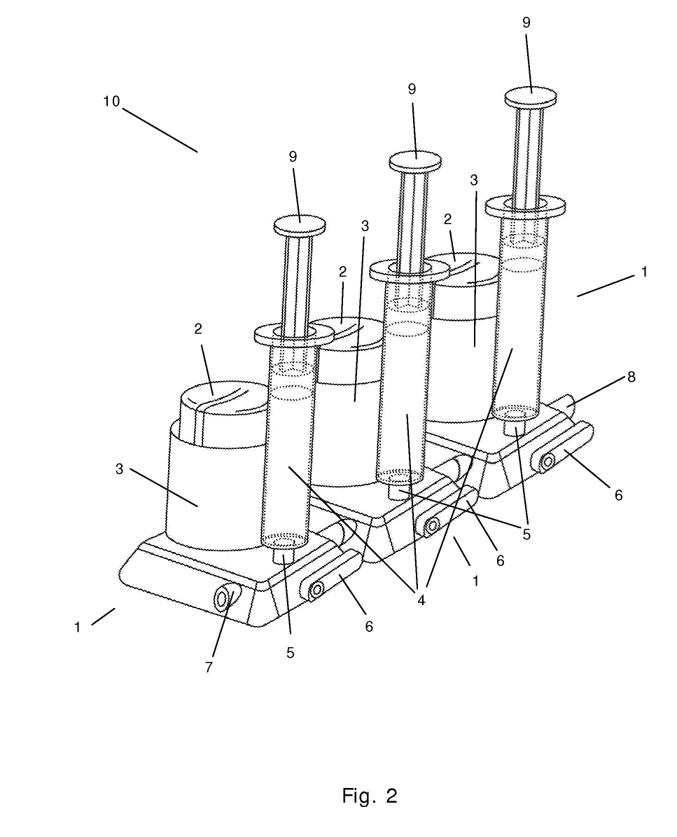

The inlet opening 8 is connectable to an outlet opening 7 of an identical device 1, and the outlet opening 7 is connectable to an inlet opening 8 of an identical device 1. Thereby a desired number of identical devices 1 can be connected in series via their inlet openings 8 and outlet openings 7. This will be described further below with reference to FIG. 2.

The device 1 of FIG. 1 may advantageously be operated in the following manner. The device 1 is preferably delivered as shown in FIG. 1, i.e. with the syringe 4 containing the liquid positioned in the syringe adapter 5, and the vial 2 containing the dry drug positioned in the vial adapter 3. Alternatively the vial 2 and/or the syringe 4 may be delivered separately, and the user may then have to position the vial 2 and/or the syringe 4 in the relevant adapter 3, 5 before use of the device 1.

When it is desired to deliver the drug of the device 1, the selector 6 is set in such a manner that a fluid connection is established between the syringe 4 and the vial 2. A movable piston 9 of the syringe 4 is then pressed in order to force the liquid from the syringe 4 to the vial 2, via the established fluid connection. The dry drug in the vial 2 is then allowed to reconstitute, and subsequently the movable piston 9 is pulled back, thereby forcing the reconstituted drug back into the syringe 4. The drug is now ready to be delivered. This is done by setting the selector 6 to the position where a fluid connection is established between the syringe 4 and the outlet opening 7, and by subsequently pressing the movable piston 9, thereby forcing the reconstituted drug towards the outlet opening 7 for delivery.

FIG. 2 is a perspective view of a device assembly 10 according to an embodiment of the invention. The device assembly 10 comprises three devices 1 identical to the one shown in FIG. 1. The three devices 1 are interconnected as described above via their inlet openings 8 and outlet openings 7. It is possible to attach an infusion device, e.g. a tube with a butterfly needle attached thereto, to the outlet opening 7 which is not connected to an inlet opening 8 of one of the other devices 1. Thus, when the reconstituted drug from each of the vials 2 has been retrieved to the corresponding syringes 4, a dose of reconstituted drug corresponding to the combined drug contained in all three vials 2 can be delivered to a person via this outlet opening 7. This may either be done sequentially, i.e. the user presses the pistons 9 of the syringes 4 sequentially in an order selected by the user, thereby delivering the drug of one vial 2 at a time, or it may be done simultaneously, i.e. the user presses the three pistons 9 simultaneously, thereby delivering the entire dose from all three vials 2 at the same time. Thus, using the device assembly 10 shown in FIG. 2 it is possible to deliver a dose of drug originating from three different vials 2 without having to move tubing attached to an infusion needle to another device, and without having to collect the drug from the three vials 2 to a common reservoir or syringe. Furthermore, it is possible to interconnect as many individual devices 1 as desired, and the individual devices 1 can be used in a normal manner without being connected to one or more other devices 1. Thereby the system is very flexible.

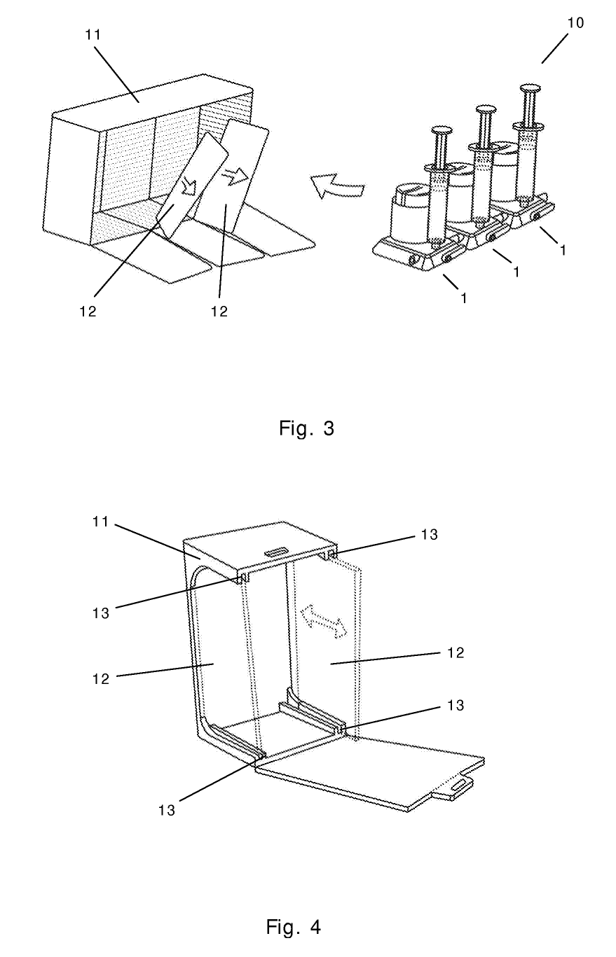

FIG. 3 is a perspective view of a device assembly 10 according to an embodiment of the invention, and of a box 11 for storing the device assembly 10. The box 11 is sufficiently large to accommodate three devices 1. It is provided with two removable walls 12. When the device assembly 10 is delivered in the box 11, the walls 12 will be inserted, and the individual devices 1 are not interconnected. When it is desired to deliver a dose of drug, the box 11 is opened, and the necessary number of devices 1 are removed from the box 11 and connected to form a device assembly 10. The dose of drug is then delivered as described above. After use the removable walls 12 can be removed. Thereby the interior of the box 11 is one large volume which is sufficiently large to accommodate the three interconnected devices 1 forming the device assembly 10. The used device assembly 10 may thereby be positioned in the box 11, and the box 11 containing the used device assembly 10 can be discarded. The box 11 shown in FIG. 3 is a cardboard box 11, and the walls 12 are removable by tearing the walls 12 along pre-punctured lines.

FIG. 4 shows an alternative box 11 for storing a device. The box 11 is connectable to another identical box 11. Thereby it is possible to construct one large box 11 similar to the one shown in FIG. 3. The box 11 of FIG. 4 is made from a plastic material. The walls 12 are removable by sliding them along holders 13.

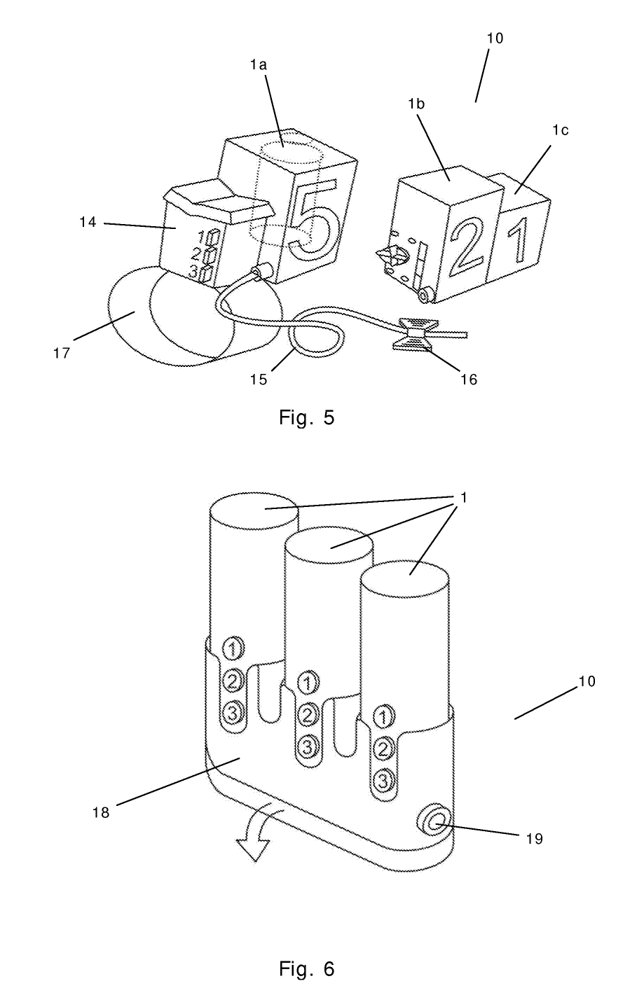

FIG. 5 is a perspective view of a device assembly 10 according to an embodiment of the invention. The device assembly 10 comprises three individual devices 1. One of the devices 1a is coupled to an operating unit 14. Furthermore, a tube 15 is connected to an outlet opening 7 of the device 1a. The tube 15 is further connected to a butterfly needle 16 for penetrating the skin of a person to receive the drug. Two additional individual devices 1b, 1c are interconnected, but detached from device 1a. The operating unit 14 may be attached to the arm of a person to receive the drug by means of armlet 17.

The device assembly 10 of FIG. 5 may advantageously be operated in the following manner. The desired number of individual devices 1 are connected as described above, and one of the devices 1a is connected to the operating unit 14. The operating unit 14 is provided with a motor for operating pistons or pumps positioned in the individual devices 1, i.e. the motor drives liquid drug out of the devices 1 and into the tube 15. The device assembly 10 is then connected to the person to receive the drug via the tube 15 and the butterfly needle 16. The operating unit 14 is switched on, thereby causing liquid drug from the devices 1 to be delivered to the person.

FIG. 6 is a perspective view of a device assembly 10 according to an alternative embodiment of the invention. The device assembly 10 comprises three individual devices 1, each positioned in a manifold 18. The manifold 18 establishes a fluid connection between the reservoirs of each of the devices 1 and a common outlet 19. Delivery means, such as a tube connected to a needle, e.g. a butterfly needle, a common reservoir, etc. may be attached to the common outlet 19. Thereby a dose of drug corresponding to the combined contents of the three individual devices 1 can be delivered from the device assembly 10 without having to move the delivery means, e.g. between the outlets of two or more individual devices 1. In the embodiment shown in FIG. 6 the devices 1 are, thus, connectable via the manifold 18.

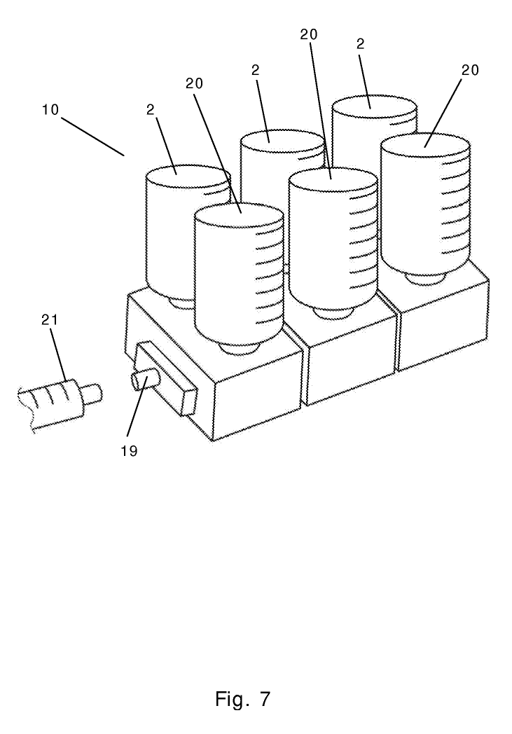

FIG. 7 is a perspective view of a device assembly 10 according to yet another embodiment of the invention. The device assembly 10 comprises three vials 2 containing dry drug and three reservoirs 20 containing liquid. The dry drug is reconstituted by forcing liquid from a reservoir 20 into a corresponding vial 2 by pressure provided in the reservoir 20 and/or by vacuum provided in the vial 2. Alternatively, the liquid can be forced into the vials 2 via a syringe 21, which is connected to a common outlet 19. A common fluid connection may establish connection between the common outlet 19 and each of the reservoirs 20 and vials 2. Thus, it is possible for the user to reconstitute from three vials 2 by use of one single device assembly 10 and via one common outlet 19 with only one syringe 20.

1. A device for delivering a liquid drug to a user, the device comprising:
a reservoir adapted to contain a liquid,
a container containing a dry drug,
means for establishing a fluid connection between the container and the reservoir in order to allow liquid to pass from the reservoir to the container in order to provide liquid drug,
an outlet opening for delivering liquid drug from the device, and
means for connecting the device to at least one other at least substantially identical device in such a manner that each of the devices is in fluid communication with a common outlet, said common outlet thereby being adapted to deliver liquid drug from each of the devices.

2. A device according to claim 1, further comprising an inlet opening being connectable to an outlet opening of another at least substantially identical device.

3. A device according to claim 1, further comprising means for connecting the device to an external operating unit, and means for transferring operational forces from the external operating unit to another at least substantially identical device connected to the device, the external operating unit thereby being capable of operating the device as well as the device connected thereto.

4. A device according to claim 3, wherein the means for transferring operational forces comprises a mechanical, pneumatic or hydraulic force transferring mechanism.

5. A device according to claim 3, wherein the means for transferring operational forces is adapted to transfer a torque.

6. A device according to claim 5, wherein the means for transferring operational forces comprises at least one drive shaft being adapted to engage with a drive shaft positioned on another at least substantially identical device.

7. A device according to claim 3, wherein the external operating unit comprises a motor.

8. A device assembly comprising at least two devices according to claim 1, said devices being interconnected in such a manner that liquid drug can be delivered from each of the devices via a common outlet opening.

9. A device assembly according to claim 8, wherein the devices are connected in series.

10. A device assembly according to claim 8, further comprising means for connecting to an external operating unit, and wherein the connection between the devices comprises means for transferring operational forces between the devices, an operational force from the external operating unit thereby being transferable to each of the devices.

11. A device assembly according to claim 8, further comprising drug delivery means for delivering a drug from the common outlet to a user.

12. A device assembly according to claim 11, wherein the drug delivery means comprises a common reservoir being detachably mounted at the common outlet.

13. A device according to claim 2, further comprising means for connecting the device to an external operating unit, and means for transferring operational forces from the external operating unit to another at least substantially identical device connected to the device, the external operating unit thereby being capable of operating the device as well as the device connected thereto.

14. A device according to claim 4, wherein the means for transferring operational forces is adapted to transfer a torque.

15. A device according to claim 4, wherein the external operating unit comprises a motor.

16. A device according to claim 5, wherein the external operating unit comprises a motor.

17. A device according to claim 6, wherein the external operating unit comprises a motor.

18. A device according to claim 14, wherein the external operating unit comprises a motor.

19. A device assembly according to claim 9, further comprising means for connecting to an external operating unit, and wherein the connection between the devices comprises means for transferring operational forces between the devices, an operational force from the external operating unit thereby being transferable to each of the devices.

20. A device assembly according to claim 9, further comprising drug delivery means for delivering a drug from the common outlet to a user.
